(19) United States
(12) Patent Application Publication
(10) Pub. No.: US 2015/0101604 A1
(43) Pub. Date: Apr. 16, 2015

(54) Nitric Oxide Generator and Inhaler

(71) Applicant: David Bruce Crosbie, Somervile, MA (US)

(72) Inventor: David Bruce Crosbie, Somervile, MA (US)

(21) Appl. No.: 14/555,559

(22) Filed: Nov. 26, 2014

Related U.S. Application Data
(63) Continuation-in-part of application No. PCT/US2013/042975, filed on May 29, 2013.

(51) Int. Cl. A61M 15/00; A61M 16/12; A61M 16/10; A61M 16/00; A61M 16/06

(52) U.S. Cl. CPC A61M 15/0086; A61M 16/0003; A61M 16/0666; A61M 16/1005; A61M 16/105; A61M 16/122; A61M 2016/0027; A61M 2016/1025; A61M 2202/0275; A61M 2202/0283; A61M 2205/3317; A61M 2205/3331; A61M 2205/3327; A61M 2205/50; A61M 2209/01; A61M 2209/04
USPC 128/203.14

(57) ABSTRACT

Several embodiments of a Nitric Oxide Inhaler that uses an electrical spark to produce Nitric Oxide from Air, optimized to maximize the production of Nitric Oxide and minimize the production of Nitrogen Dioxide through hardware and control system. Further disclosed is a system to control such inhalers.

### FIELD OF THE INVENTION

The present inventions relates generally to medical devices, and more particularly to devices for producing nitric oxide.

### BACKGROUND OF THE INVENTION

Inhaled nitric oxide (NO) is a selective pulmonary vasodilator (relaxes smooth muscle) approved by the FDA for use in the treatment of infants with pulmonary hypertension and treatment of adults with acute respiratory distress syndrome. It is also used for wound healing, and there is evidence that it is helpful in the treatment of cerebral malaria. It is also an effective treatment of Presistent Puolmunary Hypertension of the Newborn (PPHN), as well as for several other diseases.

For brevity these specifications will interchangably use the notation of capital letters NO and NO₂ to denote nitric oxide and nitrogen dioxide, respectively.

Inhaled NO is administered using NO that is transported in gas cylinders where the NO is diluted to 800 ppm by nitrogen. Significant additional equipment, with the accompanying additional cost, is required to administer and monitor the gas treatment. On the other hand, nitric oxide (NO₂) is a gas that can easily turn into nitric acid and its inhalation should, generally, be minimized or avoided.

On January 2000 the US FDA set out its guidance for a nitric oxide delivery system that delivered a steady flow of gas with a constant proportion of NO per liter of gas. It required the use of three devices which can be separately manufactured. These are: a nitric oxide delivery apparatus, nitric oxide analyzer, and nitrogen dioxide analyzer. Following these guidelines allowed for the equipment used for the administration of inhaled nitric oxide to be reclassified from class III to a class II. However further experiments has shown the benefits of delivery of NO during early inhalation—which delivers the NO to the well ventilated lung regions and reduces the delivery to the anatomic dead space. This reduces the amount of NO required, and also the amount of NO and NO₂ exhaled.

Calibration of NO delivery systems is a major factor in the cost and dissemination of NO delivery systems. The calibration is required because there is a risk with bottle based NO delivery systems that they malfunction and deliver significant quantities of NO₂.

Separating nitric oxide from air using electric arcs is done according to the formula 180 kJ+N₂+O₂=2 NO, which means that the reaction requires significant energy. This energy is typically produced using an electric arc which heats air into plasma at about 3000 degrees Celsius, to break the very strong Nitrogen bonds. Once the arc has been created, the air between the electrodes is ionized and becomes a cold plasma because only a small fraction of the gas molecules are ionized. Even as cold plasma, the electron temperature is typically several thousand degrees. The highly excited electrons collide with the oxygen and nitrogen molecules and break the bonds, enabling the production of Nitric Oxide and Ozone. The high electron mobility in the plasma reduces resistivity and the power consumption rises to the capacity of the power supply. Under a given set of conditions, the higher the supplied energy, the hotter the plasma, however the plasma is cooled by increased airflow. Arcs tend to be hottest in their center and cooler closer to the electrodes. So clearly there is no one temperature for the plasma but rather a distribution of temperatures.

The temperature of the arc determines which gases are produced. According to the US National Bureau of Standards (NSRDS-NBS 31), the dissociation energy at 300 K is approximately 945 kJ/mol for Nitrogen, 485 kJ/mol for water, and 498 kJ/mol for Oxygen. NO is formed at approximately 3,000 degrees Celsius and becomes stable at approximately 800 degree Celsius.

A continuous arc approach to Nitric Oxide generation produces significant amounts of Nitrogen Dioxide which reacts with water to form harmful Nitric acid. Human consumption of gas produced by continuous arc is preferably filtered to reduce the NO₂.

The amount of required Nitric Oxide depends on the patient needs and the amount of NO wasted. The former depends on efficiently delivering the Nitric Oxide to the patient, and the later depends on producing Nitric Oxide in a timely fashion when it is required. Ideally Nitric Oxide should be generated and delivered at the beginning of the inhalation cycle such that it will stay in the lung the longest, and the NO generation should stop before the end of the inhalation cycle.

There are three major routes of delivering NO enriched air to a patient. They will be referred to in general terms as ‘inline’, ‘injection’, and ‘standalone’, systems. Both inline and injection systems are commonly used with gas supply systems or with mechanical ventilation devices which provide air and/or gas, supplied to the patient via hoses. The usage of mechanical ventilators to assist a patients' breathing or for providing desired gas mixture is commonplace, and is generally performed by a face or nasal mask. In an inline system the inhaler is inserted in the patients' airway, i.e. between the ventilator or gas source and the patient, such that at least a portion of the ventilator/gas supply input passes through the inhaler. In an injection system the NO or the NO enriched air is injected into the oxygen enriched gas supply to the patient. A standalone system is utilized by the patient and dispenses with the ventilator and/or gas supply. Notably injected and standalone systems may utilize forced air, or suction induced directly or indirectly by the patients' breathing. Standalone systems may be easily converted to into injection systems by providing fluid coupling for the NO or NO enriched air into the air or gas path of the patient.

U.S. Pat. Nos. 7,560,076, 8,226,916, and 8,083,997 all to Rounbehler et al. provide apparatus and method for converting NO₂ to NO, by providing a delivery system that converts nitrogen dioxide to nitric oxide employing a surface-active material, such as silica gel, coated with an aqueous solution of antioxidant, such as ascorbic acid

Onkocet Ltd of Pezinok, Slovania, markets a device under the trade name Plason NO-Therapy, which uses a microwave based device to produce large flows of NO for wound healing.

U.S. Pat. No. 5,396,882 to Zapol discloses generation of nitric oxide from air for medical uses, an electric arc to create NO from air, using an electric arc chamber with electrodes separated by an air gap. An electric circuit provides a high voltage potential to the electrodes and induces electric arc discharge, which in turn produces nitric oxide mixed with air. But the solution described is expensive to produce, requires a significant amount of power, consumables, and auxiliary equipment such as pumps and monitors to operate, and moreover, in use requires expansive calibration and extensive human monitoring.

INOPulseDelivery System® (Ikaria, Inc., Hampton N.J., US), provides a system which does not contain gas measurement systems but rather delivers a preset quantity of NO (measured in moles) per breath from a gas cylinder with 800 ppm of NO. The size of each dose is dependent on the patient body mass and the breathing rate.

Therefore, there is a clear, yet heretofore unmet need, for an affordable, reliable, and power efficient nitric oxide generator. Preferably such device will also be quite, easy to clean and operate, and preferably with little or no consumables use.

### SUMMARY OF THE INVENTION

It is an object of the invention to provide a nitric oxide inhaler that produces controlled levels of NO while minimizing production of NO₂. Further objects of the invention include providing a relatively low cost inhaler that will preferably allow automatic operation, will not require frequent calibration, and that will be coupleable to a wide range of ventilator technologies.

Therefore it is an object of the invention to control NO production by controlling spark intensity and/or duration. Further optional object is to adjust the sparks responsive airflow about the spark gap. It is yet another optional objective of the invention to adjust production of NO responsive to a patients' blood chemistry parameters, such as oxygen level, methemoglobin levels, and the like.

In its most basic embodiment, the invention utilizes a controlled intensity electric arc which is created in air. The energy provided by the arc converts a portion of the air into nitric oxide, which is combined with the air to form NO enriched air. The arc is divided to a plurality of short duration arcs, also known as sparks. The term short duration in this context implies that there are a plurality of sparks per breathing cycle of a patient receiving the NO.

A spark intensity sensor is used in a feedback loop which controls the spark intensity, either of the individual spark being sensed, and/or of subsequent sparks. In some embodiments the arc is pulsed, forming short duration sparks, in some embodiments the arc is continuous, and in some embodiments a combination is used. In some pulsed arc embodiments, the sensing of the intensity of one spark is utilized to control the intensity of subsequent spark or sparks. The feedback loop enables sufficiently high precision of the NO production, to even allow automatic operation of the inhaler.

Therefore, in one aspect of the invention there is provided an air and Nitric Oxide mixture inhaler having an input and an output, the input being in communication with air, the inhaler comprising a spark chamber; at least two electrodes disposed within the spark chamber, the space between the electrodes forming a first spark gap; a spark generator electrically coupled to the spark gap, the spark generator being capable of supplying controlled amount of electrical energy to the spark gap; a controller coupled to the spark generator; a spark intensity sensor optically coupled to the spark gap, the sensor being coupled to the controller. During operation the electrical energy supplied to the electrodes is sufficient to cause a plurality of sparks across the spark gap at intervals controlled by the controller, the controller is further configured to control energy supplied to the spark responsive to information received at least from the spark intensity sensor, and the spark energy is directed to enrich air with nitric oxide, the nitric oxide being produced from the air by the spark.

Optionally a third electrode is also provided, forming a second spark gap between the third electrode and one of the at least two electrodes. More spark gaps may be utilized, and the selection of the number and arrangement of spark gaps is a matter of technical choice.

Further optionally, the controller is capable of tracking the number and the intensity of a plurality of sparks generated between the at least two electrodes over a period of time. In an inhaler which has a plurality of spark gaps, the controller is optionally capable of tracking the number and intensity of sparks generated in the plurality of spark gaps over a period of time.

Optionally any of the inhaler embodiments further comprises an input for receiving information from an oximeter, and the controller adjusts the production of nitric oxide in response to the information from the oximeter. The oximeter input may be direct or indirect via a second device, a communication link, and the like.

The controller may be configured to collect information about the flow of energy directed to at least one electrode, and to estimate the condition of the at least one of the electrodes by the distribution of the energy flow. Alternatively or additionally, the controller may further utilize information from the spark intensity sensor to estimate the condition of the at least one electrode.

Optionally the inhaler further comprises at least one treatment profile, wherein the controller is configured to control the inhaler according to the at least one treatment profile. In certain embodiments the profile may be stored on a different device and communicated as a whole or in part to the inhaler. By way of example the profile comprises at least one element of a list of elements consisting of nitric oxide quantity per treatment, nitric oxide delivery rate per unit time, nitric oxide generation profile per breath cycle, nitric oxide generation responsive to information about one or more patient parameters, treatment duration, treatment cycle, nitric oxide generation responsive to environmental parameters, nitric oxide generation responsive to airflow in spark chamber, and any combination thereof.

In certain embodiments, the inhaler further comprises a membrane disposed to receive the nitric oxide enriched air on one side, the membrane being permeable to nitric oxide, and impermeable to nitric oxide.

In certain embodiments generally referred to as inline type, the input and the output are disposed in an air path of a patient. In other embodiments, generally referred to as standalone type and/or injector type, the output is in fluid communication with an air path of a patient. By way of example, such fluid communication may be direct link to the patients' mouth, to a mask, a cannula or the NO may be injected to a regular airway such as be coupled to the hoses leading from the hospital gas supply to the patient, but in standalone embodiments the input to the inhaler is separate from the patients air path.

Optionally the inhaler further comprises an air velocity sensor disposed to sense air velocity within the spark chamber, the air velocity sensor being coupled directly or indirectly to the controller, and the controller is being configured to adjust the spark energy responsive to the velocity of air within the spark chamber.

Optionally, the inhaler may further comprise an orifice coupled to a forced air supply, the orifice being directed such that air passing therethrough is directed at the spark gap. If there is more than one spark gap then optionally the air is directed at more than one spark gap via one or more orifices.

Further optionally, the inhaler comprises an inhalation sensor disposed to sense inhalation by a patient receiving the nitric oxide produced by the sparks. In such embodiments, production of sparks optionally occurs responsive to information from the inhalation sensor. In certain embodiments the controller starts the creation of nitric oxide in synchronization with the patient's breathing cycle. Optionally, the inhaler is configured to deliver a predetermined amount of nitric oxide during each breath cycle.

In certain embodiments the controller is configured to vary the amount of produced nitric oxide over a period of time. The period of time may range over more than a single time range. Thus by way of example the controller may change the production of nitric oxide over the period of a single breath cycle, but it also may change the total amount of NO from hour to hour, and similar combinations. The change may occur responsive to pre-programming a treatment schedule, in response to patient or medical provider input, in response to sensed parameters, such as air velocity, or any combination thereof.

Optionally the inhaler further comprises a data link. The link may be wired or wireless, and may be utilized for programming and/or controlling the inhaler, or for providing information thereto or obtaining information therefrom.

Optionally the controller is configured to receive information from at least one sensor selected from an oximeter, methemoglobin sensor, a blood parameter sensor, an environment sensor, and a breathing volume sensor. A combination of any of the above mentioned sensors is also considered. The sensor or sensors may be wired to the device or received via the optional data link if one is provided.

Optionally the inhaler comprises an input for inputting information relating to at least one dynamic parameter of a patient receiving the produced nitric oxide, and wherein the controller is configured to control the amount of produced nitric oxide in response to the information. By way of example, the dynamic parameter may relate to methemoglobin blood level of the patient. In an optional embodiment, the methemoglobin information is derived by modulating the amount of nitric oxide produced over time and delivered to a patient, and by monitoring the response to such modulation using sensing variations in the patient's blood oxygen levels.

In another aspect of the invention, there is provided an air and Nitric Oxide mixture inhaler having an input and an output, the input being in communication with air, the inhaler comprising a spark chamber; at least two electrodes disposed within the spark chamber, the space between the electrodes forming a first spark gap; a spark generator electrically coupled to the spark gap, the spark generator being capable of supplying controlled amount of electrical energy to the spark gap; a controller coupled to the spark generator, wherein during operation the electrical energy supplied to the electrodes is sufficient to cause a plurality of sparks across the spark gap at intervals controlled by the controller, the controller is further configured to control the energy of the sparks; an inhalation sensor coupled to the controller, and disposed to sense inhalation by the patient. The sparks energy is directed to enrich air with nitric oxide, the nitric oxide being produced from the air by the spark, the nitric oxide being supplied to a patient, and the controller is configured to cause generation of nitric oxide during inhalation of the patient.

In yet another embodiment there is provided an air and Nitric Oxide mixture inhaler having an input and an output, the input being in communication with air, the inhaler comprising a spark chamber; at least two electrodes disposed within the spark chamber, the space between the electrodes forming a first spark gap; a spark generator electrically coupled to the spark gap, the spark generator being capable of supplying controlled amount of electrical energy to the spark gap; a controller coupled to the spark generator, wherein during operation the electrical energy supplied to the electrodes is sufficient to cause a plurality of sparks across the spark gap at intervals controlled by the controller, the controller is further configured to control the energy of the sparks. The controller is configured to receive information from a sensor measuring at least one parameter of a patient, and adjust the nitric oxide production in accordance with at least the one parameter. In certain embodiments the sensor is an oximeter. The sensor may be wired directly to the inhaler or communicate via an optional data link or via a third device such as a computer and the like.

In yet another aspect of the invention, there is provided an air and Nitric Oxide mixture inhaler having an input and an output, the input being in communication with air, the inhaler comprising a spark chamber; at least two electrodes disposed within the spark chamber, the space between the electrodes forming a first spark gap; a spark generator electrically coupled to the spark gap, the spark generator being capable of supplying controlled amount of electrical energy to the spark gap; wherein during operation the electrical energy supplied to the electrodes is sufficient to cause a plurality of sparks across the spark gap at intervals controlled by a controller. The inhaler further comprises a data link configured to communicate at least with a computer and receive operating parameters therefrom. In certain embodiments the controller is disposed within the controller or coupled directly thereto, and on other embodiments the controller, or portions thereof, may be disposed remotely and communicate with the inhaler via the data link. In a related aspect there is provided a system for administering controlled amounts of NO to at least one patient, the system comprising a computer having a data link, the data link being in data communication with an inhaler capable of providing controlled amount of NO to a patient, the inhaler further having a data link for communicating with the computer. In preferred embodiments, the inhaler conforms to any of the embodiments described herein. Furthermore, the system may control a plurality of inhalers, for administering controlled amounts of NO to a plurality of patients.

Similar combinations of the features described above may be incorporated in embodiments of different aspects of the invention, as will be clear to the skilled in the art in view of the teachings presented herein.

### SHORT DESCRIPTION OF DRAWINGS

The summary above, and the following detailed description will be better understood in view of the enclosed drawings which depict details of preferred embodiments. It should however be noted that the invention is not limited to the precise arrangement shown in the drawings and that the drawings are provided merely as examples.

FIG. 1 depicts an external view of an online type inhaler embodiment.

FIG. 2 depicts a view of a standalone type inhaler embodiment.

FIG. 3 represents a simplified block diagram of components of a inhaler, showing the basic, and some optional, components.

FIG. 4 depicts a simplified flow diagram of basic and some optional operations of a controller in accordance with some embodiments.

FIG. 5 depicts a simplified flow diagram of a method to determine methemoglobin in a patient's blood.

FIG. 6 depicts a system for administering a NO to a plurality of patients

FIG. 7 depicts a spark chamber having a plurality of spark gaps and optional nozzles.

FIG. 8 depicts yet another embodiment depicting use of selective membrane for reducing NO₂ inhalation.

### DETAILED DESCRIPTION

Certain exemplary embodiments of the invention will now be described, to facilitate better understanding of the basic, as well as the many optional components and features provided by different aspects of the invention. The description is provided by way of example only and not all elements are required for proper operation of the invention. Therefore the examples should be construed broadly as showing the myriad of possible extensions, rather than as limiting the scope of the invention.

Sparks are used to generate the NO from the air are created between at least one pair of electrodes forming a spark gap. In some embodiments a plurality of sparks are formed between a plurality of electrodes. The power level supplied to the electrode and/or the duration of the sparks may be controllably varied. While for brevity and readability most of these specifications will describe the operation of a single spark gap, it is specifically noted that a plurality of electrode, forming a plurality of spark gaps is considered and the specifications and claims should be construed to extend to such embodiments. In certain multiple spark gap embodiments spark are created simultaneously, while in other embodiments they may be spread in time between different spark gaps.

The terms ‘spark’ and ‘arc’ are used interchangeably in these specifications, as a spark is an electrical arc having a short duration. For the purpose of these specifications, a short duration is considered to span a time shorter than breathing cycle of a patient receiving the NO generated by the spark, however spark duration is commonly far shorter and typically extends in the order of milliseconds to a few seconds.

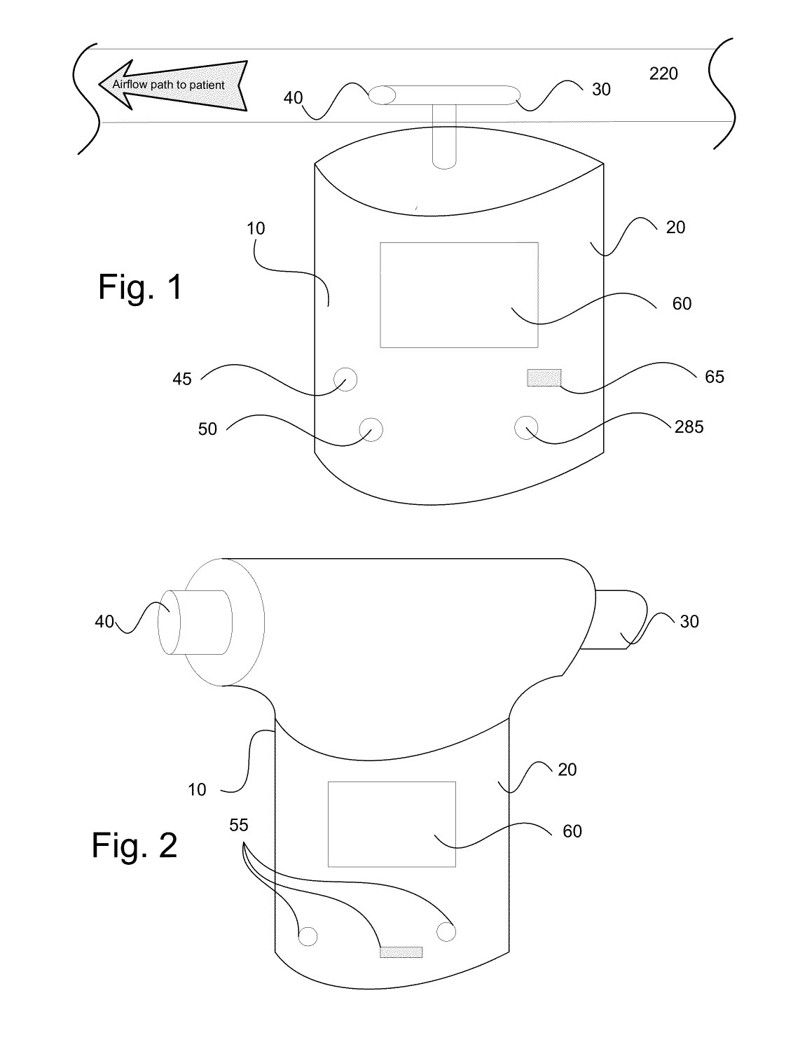

FIG. 1 depicts a general view of an inhaler 10 belonging to one family of embodiments of the invention that will generally be referred to as an online type. Such online type is generally coupled to an airway 220 of a patient, such as a mask hose connecting a patient to a mechanical ventilation device or gas supply. The inhaler has an enclosure 20, an air inlet 30, and an NO enriched air outlet 40. The inlet and outlet are inserted into the patient's airway. In contrast, FIG. 2 depicts a inhaler which will be referred to as a standalone inhaler. The standalone inhaler may be utilized as a separate inhaler, or the output may be coupled to an airway such as a cannula or mask hose. In a standalone device only the outlet 40 need to couple to an injector port of a patient's airway 220 if one is used. If the device is used indeed as a standalone device, the patient may inhale directly from the inhaler outlet. Notably, in the online device a second inlet may be used for air to be converted to NO (not shown).

A power supply connection 45 and an optional oximeter input 50 are typically also provided. The inhaler also has an optional input device 55 for allowing user input and a display 60, which may comprise indicator lights, alphanumeric display, and the like. Alternatively and/or additionally, the inhaler may communicate an external device such as a computer or specialized controller using a data link. Optionally, a data connection 65 is supplied for wired communication links. A wireless connection may be utilized, and such connection may be utilized instead of, or in addition to, the data connection 65. The input device may comprise one or more buttons as shown in FIG. 2, it may be a detachable device, or may be remotely coupled via the data link.

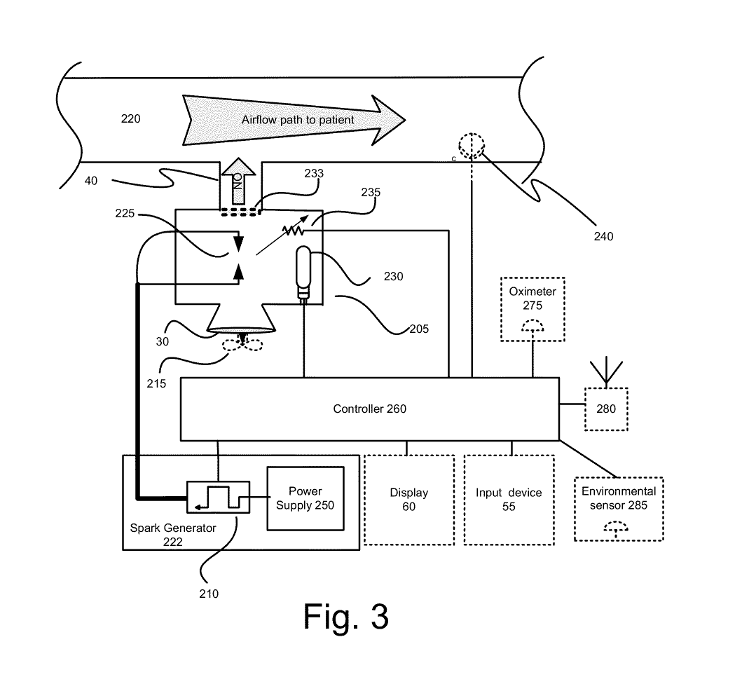

FIG. 3 represents a simplified block diagram of components of a inhaler, showing the basic, and some optional, components. While this drawings depicts a standalone type, the skilled in the art would readily see how the design may be adapted for inline type device by locating the air input in the patient air path. 220. Spark chamber 205 contains at least one pair of electrodes forming a spark gap 225 therebetween. Air is entered into the spark chamber via inlet 30, and NO enriched air is injected from the output 40 into the gas flow to the patient's airway 220. In some embodiments, a filter 233 is utilized to filter NO₂ molecules. In certain embodiments air is forced into the chamber 205 by a fan, an air pump, or any other forced air source 215. Optionally, an inhalation sensor 240 is disposed to sense airflow in the airway. More particularly, the inhalation sensor is capable of differentiating between inhalation and exhalation.

A spark intensity sensor 230 is disposed to sense the intensity of sparks. The spark intensity sensor is utilized as a portion of a feedback loop controlling the amount of NO produced by monitoring the spark intensity.

The spark gap is electrically coupled to a spark generator 222. Spark generator 222 comprises circuitry for generating the spark and for controlling the energy supplied to the spark gap. If a plurality of electrodes are used within the spark chamber, the sparks may be distributed between the spark gaps by distributing striking of sparks across one or more spark gaps, and the spark generator controls the energy distribution to the plurality of the spark gaps. Each spark creates a known molar quantity of NO, and by varying the quantity with the patient's breathing cycle, a pulse of NO can be delivered with high precision as the dose can be changed every few milliseconds.

The spark generator 222 is controlled directly or indirectly by controller 260. The controller comprises logic that monitors the production of NO. The controller receives information from the spark intensity sensor 230, and completes the basic feedback loop. The basic feedback loop is formed by the striking of a spark in the spark gap, and sensing the spark intensity by the spark intensity sensor 230. The spark intensity information is transferred to the controller 260 which controls spark generator 222. Spark generator 222 adjusts either the energy to the current spark, or to future sparks, in response to the controller instruction, which are in turn the result of the information provided by the spark intensity sensor, combined with other logic which may be adjustable, or set during manufacture.

In some embodiments, an optional inhalation sensor 240 is also coupled to the controller 260. The controller utilizes information received from the inhalation sensor, and when the information indicates an inhalation, the controller commands the spark generator to start generating sparks, to cause creation of NO. In certain embodiments an optional air velocity sensor 235 is also provided to measure the air velocity to which at least one of the spark gaps is exposed. Further optionally, the controller may be coupled to an oximeter 275 which measures certain aspects of the blood chemistry of the patient. In some embodiments the controller also receives information such as ambient air temperature and humidity and the like from an external environmental sensor 285.

Optionally the controller 260 further communicates with an input device 55. The input device may comprise of buttons and/or a keyboard, or a data port, and the selection of the input device type is a matter of technical choice. Similarly, a display device 60 may also be coupled to the controller. The display device may comprise any convenient display, such as lights, and/or alphanumeric display. The optional input and output devices can provide status display, and optionally to program the device's operation. Alternatively or additionally, the inhaler 10 may comprise a data link 280, in communication with the controller. The data link may be of any desired type. By way of example the data link may be a wired link such as USB, IEEE 1394, Ethernet, and the like. The data link may also be a wireless data link such as an IEEE 802 type Wi-Fi link, Bluetooth, Zigbee, and other low range links, a cellular link, and the like. More than one type of data links may be used in combination. A data link allows communication between the inhaler and one or more external devices. Data link may be utilized to program the inhaler, and/or receive data therefrom. The data link may also be used to communicate with an oximeter and other sensors, obviating the need for the dedicated inputs. A data link may further be used to connect to a remote display and input devices, obviating the need for the optional local input device 55 and display 60. A data link also allows controlling a plurality of inhalers from one or more remote sources, which reduces the cost of individual devices. Other information may be fed to the controller, such as gas supply information to the patient, and the like.

A pulsed arc discharge reduces the average temperature of the gas in the region of the arc, but it does not decrease the peak temperature because the thermal mass of the gas is low and the heat loss is high. Indeed each pulse results in a very high peak temperature. It is therefore desired to establish an arc and reduce the power to the arc to the point of optimum Nitric Oxide production—too low an arc temperature will favor the production of Ozone as opposed to Nitric Oxide, and too high a temperature will cause the Nitric Oxide to react with the ozone and produce Nitrogen Dioxide. Selection of the energy levels provided to the spark may be determined empirically, by numerical simulation, by approximation, by calculation, or by any combination of such methods.

If the energy per spark is kept constant (by monitoring the spark intensity and using that information to control the subsequent sparks) then there is a strong correlation between the number of sparks per second, the air flow rate, and the concentration of NO (approximately 6 mg of NO per minute per watt of power). This allows the NO production to be controlled by measuring the spark and adjusting the number of sparks per a period of time. Alternatively, it is possible to adjust the energy supplied to each spark.

The spark generator contains a high voltage power supply 250, which supplies sufficient voltage to strike an arc across the spark gap, and maintain it thereafter for a controlled period of time. In some embodiments spark generator 260 reduces the energy supplied to the arc after the arc is struck. In some embodiments the spark gap generator comprises pulsing circuitry 210 to generate a plurality of sparks by switching current to selected electrodes. In other embodiments a capacitor is charged to a desired level, and the energy in the capacitor decays over time in accordance with current flow, until such point that the voltage is insufficient to maintain the arc. In certain embodiments, such as those using a Marx generator described below, the spark duration is controlled by the structure of the Marx generator. In some embodiments a capacitor is charged and the spark duration is a function of the amount of charge. In some embodiments a capacitor is discharged through a step-up transformer and into the spark gap. In certain embodiments a trigger electrode may be utilized. In other spark generator embodiments, switches, such as transistors and the like may be used to limit the spark duration. The skilled in the art would readily recognize many other methods of controlling the spark intensity.

Providing circuitry and/or structure for generating sparks and for controlling the duration and/or intensity thereof is a matter of technical choice well within the level of the skilled in the art.

Utilizing charged capacitor power supply offers a naturally diminishing spark intensity. The capacitor is charged to a pre-determined voltage, and then an electronic switch discharges the capacitor, and the energy is fed to the electrodes to cause the spark. Once a spark is struck, the capacitor begins rapid discharge, and the current is fast reduced in accordance with the reduced charge.

Low duty cycle of the sparks is advantageous. Stated differently it is desired that the dwell time between sparks is long relative to the duration of the spark. The long gap between sparks allows the gases to cool and thus reduce the creation of NO₂. To that end, utilizing a plurality of spark gaps allows staggered us of the spark gaps, offering a lower duty cycle for each individual spark gap.

This concept of multiple spark gaps can be extended using a modified version of a Marx Generator. This design uses building block, each consisting of an inductor (or high value resistor) and a capacitor in series, with a spark gap across the capacitor and inductor. A number of these building blocks are placed in parallel, and charged up. When the first spark gap ionizes then it becomes a conductor and connects the first capacitor in series with the second capacitor. The combined voltage across these two capacitors triggers the second spark gap, which connects the first two capacitors to the third capacitor, until the spark propagates through the spark gaps of all the building blocks. Charging such an array requires a high voltage power supply and time. An additional trigger electrode placed by the first spark gap may be used to trigger such a circuit. This electrode is triggered using a capacitor discharge circuit and associated circuitry.

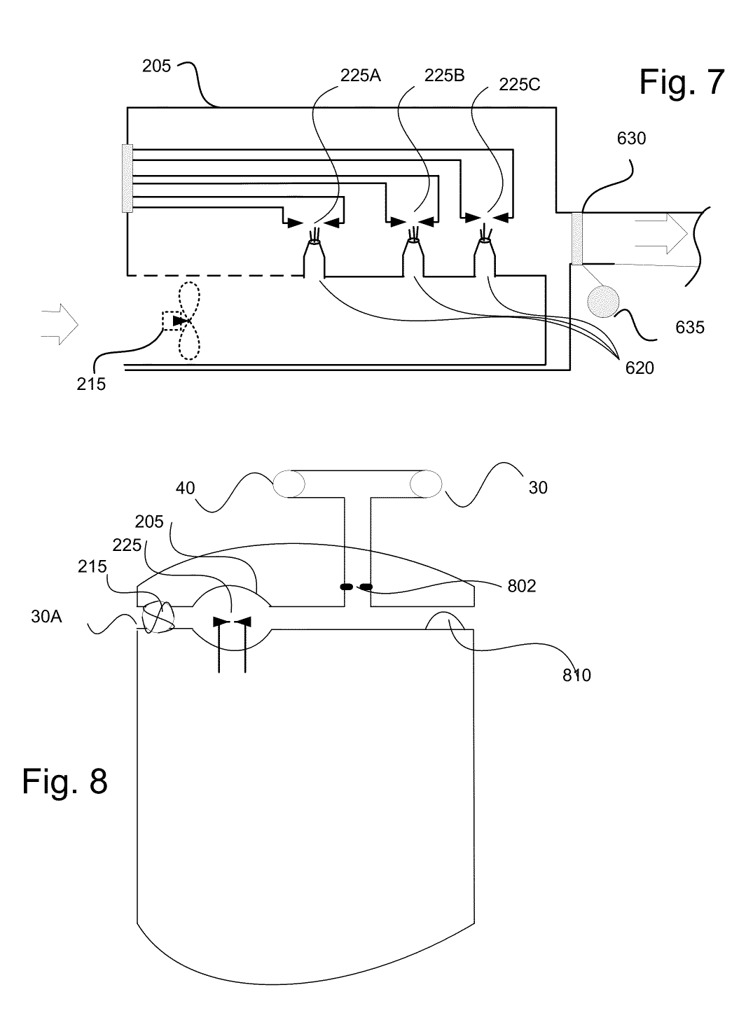

Notably in certain embodiments a plurality of spark gaps are provided Such as depicted by way of example in FIG. 7 by plurality of spark gaps 225A, 225B, and 225C. Multiple spark gaps allow better control the production of NO while reducing generation of NO₂. Utilizing a plurality of spark gaps allows reduction of the spacing of the gap, which allows generating similar amounts of NO at a lower voltages. Sparks that use such low voltage are commonly referred to as ‘micro sparks’.

Using micro sparks also facilitates reduction in the spark voltage. In some embodiments spark voltage as low as 1000V and below is utilized. Such low voltage individual sparks allow a lighter and simpler power supply. Lower voltage reduces the cost of the high voltage generator, and allows use of high voltage transistors to turn on and off the power to individual electrodes.

A spark intensity sensor 230 may measure light intensity at one or more frequencies or frequency band. Alternately the spark intensity sensor may utilize electromagnetic radiation caused by the spark. Further, a microphone may be utilized for spark intensity sensing, but such design is considered less accurate. It is well known that a microphone is a sensor sensing pressure changes, however it is noted that an absolute or relative pressure sensor may also be considered a microphone, by comparing changes over time. Therefore, such a pressure sensor may also be utilized as a spark intensity sensor, since the spark causes a distinct change in the fluid in which it sparks. Further it is possible to reduce the complexity of the device by using a single pressure sensor to determine the spark intensity as well as the airflow through the device.

While one may monitor the amount of energy supplied to the spark gap to determine the amount of NO produced, such method is exposed to inaccuracies stemming from changes in humidity, electrode condition, and the like. However monitoring the discharge voltage, and/or the strike voltage may provide an indication of the state of the electrodes, thus in some embodiments, the controller is constructed to detect the state of the electrodes by monitoring directly or indirectly the voltage supplied to the electrode, and assert an alert signal when the voltages exceed specific values, or take other corrective action. In certain embodiments such alert condition may be obtained by correlating the spark intensity and the energy supplied to the spark.

If the arc is cooled, by increasing the air flow, then the arc temperature drops, the resistance rises, and hence less power dissipated. If the air flow is sufficiently strong then all the ionized gases are removed and the arc will only reform if there is sufficient voltage across the electrodes to ionize the gases again. Higher air velocity improves both the production of NO and decreases the relative production of NO2. It was found that air velocities at or above 100 meters per second provides excellent results, but higher and lower levels are also explicitly considered, and the selection of speed is related to the overall construction of the chamber and spark gaps, as well as to the voltages applied to the spark gaps. Determination of the ideal air velocity may be determined experimentally, by calculation, simulation, and the like. In certain embodiments, a forced air source 215 (such as from a fan, a pump, the hospital air supply, and the like) is fed through the chamber 205. FIG. 7 depicts one optional feature where, air flow is aimed directly at the spark gap, such as by a properly directed narrow tube or orifice 620. Such air flow provides high air velocity and significant arc cooling. The low flow and high pressure makes it possible to inject the NO enriched air into an oxygen rich supply to a nasal cannula or face mask without significant dilution of the patient oxygen supply.

Certain types of inhalation sensors are well known in the art. By way of example, a sensitive pressure sensor may monitor a pressure drop in the patient's airway path. Another method of sensing the airflow uses hot wire anemometer, where a heated metal filament is placed in the air flow, and a drop in the temperature is detected, commonly by way of sensing changed resistance of the filament, which is followed by a change in current flowing therethrough. By placing a second filament in the same airflow, but behind a wind shield, it is possible to differentiate between inhalation and exhalation. Yet another inhalation sensor utilizes a microphone situated close to the patient's mouth and nose. The sound of inhalation and exhalation is distinct, and the depth of each breath can also be estimated. Similar solutions may be utilized to provide sensing of airflow through the spark chamber.

Various patient parameters sensors are known in the art. By way of example, oximeters, and other blood chemistry sensors are well known. Similarly, lung capacity and tidal volume of the patient may be sensed. Environmental parameters such as temperature, pressure and humidity are in common use. Other sensors may be utilized as desired, to be considered in the treatment profile directed to the patient. Such profile may include such parameters as simply the total effort to generate NO during the treatment—setting the sparks energy level and allowing the inhaler to run for a prescribed period of time. While such operation is considered, the advantages of the supplied logic provide better options. In the embodiments where a spark intensity sensor is provided, the actual production of NO may be closely monitored, and the spark intensity is closely correlated to the amount of NO produced. Treatment profiles which consider static parameters of the patient, such as age, sex, weight, specific disease, other known conditions, and the like, may be provided. Monitoring of dynamic parameters such as blood chemistry, pulmonary function, motion, and the like may be done, and treatment profiles may be selected or adjusted to accommodate such changing conditions. Time dependent profiles, such as providing NO at intervals, or varying the amount of NO administered may over time may also be dictated by the treatment profiles. Profiles may be adjusted according to past history of prior treatment. Profiles may be set by the manufacturer, set according to a patient specific prescription, or a combination between the two options, where the inhaler is pre-programmed and adjustments to the program are created to provide best fit to each patient needs.

In some embodiments, the inhaler further comprises a humidifier 630 disposed to receive the nitric oxide enriched air. Utilizing the humidifier causes at least some NO₂ which is created by the sparks to be absorbed in the water vapor, and form a mild acid, which is then collected, such as by reservoir 635.

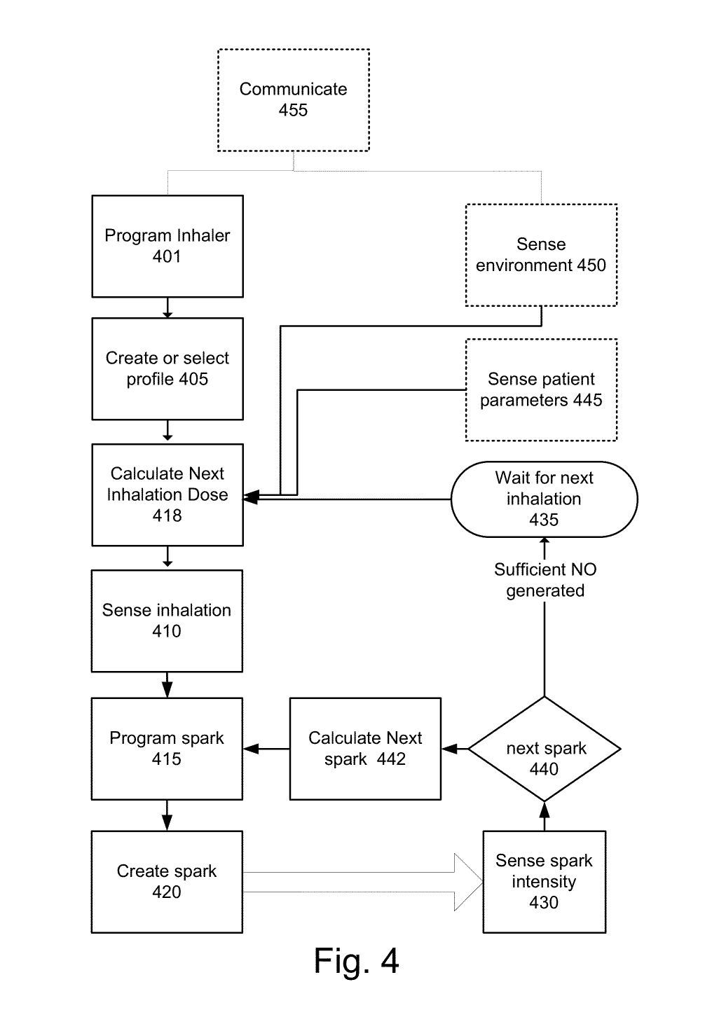

FIG. 4 depicts a simplified flow diagram of various modes and options of operation of different aspects of a controller according to some aspects of the invention.

The inhaler comprises logic which controls aspects of its operation. While in some embodiments the logic may be fixed during manufacturing of the inhaler, in the embodiment depicted in FIG. 4 the inhaler is programmable. Programming 401 may be carried out by a separate device such as a computer, or through a local input device 55. Programming involves selecting, adjusting, or setting a profile for treatment 405. The profile dictates treatment parameters such as the amount of NO to be delivered at a particular time phase of the treatment, the duration of the treatment, how the dosage varies with time, and the like. Profiles may be pre-stored in the inhaler programmed individually for each patient, or a combination where preprogrammed profiles are adjusted to fit specific patient needs.

Next the dose for a single inhalation is calculated, and the desired NO quantity is set for the next breath cycle 418. In some embodiments NO is produced only during inhalation, and the production of NO begins after inhalation is detected 410, while in other embodiments the production on NO is continuous, and the desired dosage of NO production is calculated per unit of time or volume of inhaled gas.

In some optional modes of operation, the controller dictates generating larger amounts of NO at the initial stage of the breathing cycle, and reducing or even stopping NO production as the inhalation progresses. Such timing allows the NO to reach deep into the lung. Furthermore, it is desired to generate NO only during the inhalation, as doing so reduces the time the NO is susceptible to turning into NO₂, and be lodged in the patient's lungs.

Control of production of NO may be carried out by numerous ways that will be clear to the skilled in the art in view of the present specifications. By way of example, the amount of energy of each spark may be controlled, the duty cycle of the sparks may be adjusted, the number of spark gaps to be used in the case of a plurality of spark gaps, the duration of spark generation may be shortened or lengthened, and the like.

In the depicted example the spark is programmed 415 on a breath by breath basis according to the profile 405, with the objective of creating the target amount of NO for that breath and according to the desired NO inhalation profile. The first spark is done with a default or programmed condition. The term ‘programming the spark’ with all of its grammatical inflictions, imply selecting any number of parameters such as the number of sparks to be fired in individual spark gaps, in embodiments using a plurality of spark gaps, the length of the spark, and/or the energy level of the spark. In certain embodiments, such when a charged capacitor is used as the high voltage source for the spark, controlling the amount of energy in the capacitor is sufficient to dictate both the length of the spark and the energy level, for a given environment. In other embodiments, the spark may be ignited and then extinguished at desired time level, and the energy level is controlled separately.

The spark is then created 420.

The spark intensity is sensed 430 by the spark intensity sensor, and the information is transferred to the controller. While in some embodiments the controller adjust the spark intensity dynamically during the spark existence, it is more economical to use the information of a spark to control the intensity of subsequent spark or a plurality thereof.

If additional NO generation is desired for the present breath cycle, step 440 passes control to step 442, where the desired intensity of the next spark is calculated using information regarding previous spark intensity. In some embodiments information comparing the spark intensity to the energy provided to the spark gap is also considered during the calculation. If sufficient NO has been generated then the controller waits for the next inhalation 435 before generating the next spark. The selected profile oftentimes provides further information which is utilized during calculation of the next spark. As the treatment is generally spread over a long time with a large plurality of sparks and a large number of breathing cycles, the history of NO delivery is also considered. Optionally, further information may be derived from sensing 445 at least one parameter of the patient, such as blood chemistry, breathing cycle information, oxygen level, and the like. Further adjustments may be made if the environment is sensed 450. Environmental information such as air pressure and temperature may be utilized to better gauge the level of NO produced, while humidity sensing may dictate not only the spark intensity, but possibly whether to continue or pause a treatment. If an optional air velocity sensor 235 is used, information therefrom may also be utilized to determine spark parameters.

The feedback loop provides by 415, 420, 430 and 440 increases NO accuracy generation and minimizes the need for calibration, as the sensed spark intensity is closely correlated with the amount of nitric oxide production. This basic feedback loop may be enhanced by steps such as 435, 418, and others. The total amount of NO produced is adjusted in accordance with additional parameters, such as the profile, measurements of the patient's parameters, environmental parameters, and the like. In the depicted embodiment, no sparks are generated during exhalation, but inhalation and/or exhalation volume and duration may be monitored to obtain tidal volume and adjust the NO production according thereto. Once the next inhalation is sensed, the process begins again at stage 410, however at this time the history of NO generation and all other factors described supra may be utilized.

If the inhaler is equipped with a data link, some embodiments allow the programming of the inhaler 401 to take place utilizing 455 the data link. If desired, the data link may also be used to deliver information to a computing device coupled to the data link. Such information may, by way of example, indicate the status of the treatment, warn of malfunction, relate patient parameters such as breath rate, oxygenation levels, and the like.

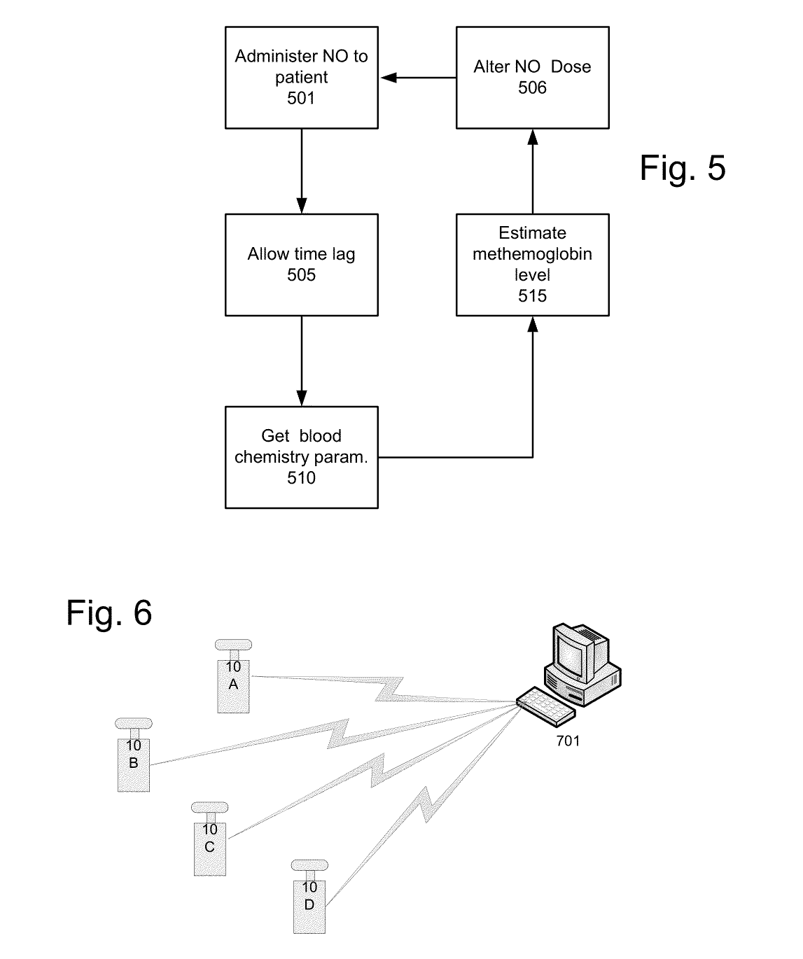

FIG. 5 depicts a simplified block diagram of a method of measuring or estimating a patient's methemoglobin. A known amount of NO is administered 501 to a patient. Optionally this amount differs from the continuous mean amount of NO administered to the patient, and the varied amount of step 501 acts as a ‘marker’ to the beginning of the measurement/estimate cycle. After a predetermined time lag 505 a measurement of at least one parameter of the patients' blood chemistry is obtained 510. By way of example the measured parameter may be oxygen level, blood hemoglobin level, and the like. By correlating the amount of administered NO and the blood chemistry after time measured after the time lag, it is possible to more precisely estimate 515 the actual level of methemoglobin in the patient's blood. Such correlation may be done by utilizing general curves related to the level of supplied NO, the time lag, and the measured oxygen. Preferably, the correlation is done by fitting certain actual measurements of blood components, such as methemoglobin levels in specific patient, to such curves, or providing sufficient measurements to create such curve per patient. The dose of NO to be administered to the patient may be adjusted 506 in accordance with the results of the estimate.

The NO inhaler may follow a dosage curve—starting with a relatively high level of NO (80 ppm is typical) and then reducing this level when the level of oxyhemoglobin or methemoglobin met certain criteria. The inhaler may show a simple health indicator to medical staff which would show the stage of the treatment, and the relative health of the patient (by way of example, high oxyhemoglobin and low methemoglobin indicates success, while the reverse may indicate that the treatment is not effective despite a high dose of NO). In some embodiments, the inhaler would automatically reduce the level of NO at the end of the treatment to mitigate withdrawal effects.

FIG. 6 depicts a system suitable for administering NO for at least one patient, and preferably to a plurality of patients, from a central control device. The central control device 701 may be a general purpose or a special purpose computer, such a s a PC, a cell phone, a tablet, or a dedicated control device. By way of example the control device may be located in a nurse station in a hospital, where a single nurse is then capable of monitoring the plurality of patients, or it may be a cellular telephone carried by the treating personnel. Selectively certain access rights may be utilized for programming and for monitoring.

FIG. 7 depicts a simplified diagram of one optional spark chamber 205 construction. This embodiment combines optional features such as a plurality of spark gaps 225 A, 225B and 225C, as well as an arrangement to increase airflow velocity about the spark gaps. Forced air source 215 which feeds air to nozzles 620 which increases the airflow velocity and reduces the creation of NO₂ by reducing the spark temperature. Yet another optional feature depicted in this embodiment is the humidifier 630 which helps removing NO₂ by combining it with the gas, and then preferably removing the condensate into collector reservoir 635.

FIG. 8 depicts yet another embodiment of the inhaler, showing yet more optional features. In this embodiment utilizes two air inlets: the regular inlet 30 and inlet 30A. While inlet 30 and outlet 40 may be placed in the patients' airway 220, air for generating NO is admitted through additional and separate input 30A. The air entering at 30A is either already at a pressure higher than the ambient pressure, or is pressurized to such level by forced air source 215. The air passes through the spark chamber 2015 and by the one or more spark gaps 225, as in the other embodiments. However instead of being simply directed to the patient, the air is released via a choke 810. The choke slows down the amount of NO enriched air exiting the inhaler. A membrane 802 is exposed to the NO enriched air and to the air or gas being inhaled by the patient. The membrane is permeable to NO but impermeable to the much larger NO₂ molecule. Thus the patient receives an excellent protection from inhaling damaging compounds.

It is important to notice that the term “air and Nitric Oxide mixture inhaler” implies that the nitric oxide is generated from the air, and it is not necessary for the air used in generating the NO to be inhaled by the patient. In some embodiments this is the case, while in others, such as those using a permeable membrane by way of example, ideally only the NO is utilized by the patient.

It will be appreciated that the invention is not limited to what has been described hereinabove merely by way of example. While there have been described what are at present considered to be the preferred embodiments of this invention, it will be obvious to those skilled in the art that various other embodiments, changes, and modifications may be made therein without departing from the spirit or scope of this invention and that it is, therefore, aimed to cover all such changes and modifications as fall within the true spirit and scope of the invention, for which letters patent is applied.

What is claimed is:

1. An air and Nitric Oxide mixture inhaler having an input and an output, the input being in communication with air, the inhaler comprising:
a spark chamber;
at least two electrodes disposed within the spark chamber, the space between the electrodes forming a first spark gap;
a spark generator electrically coupled to the spark gap, the spark generator being capable of supplying controlled amount of electrical energy to the spark gap;
a controller coupled to the spark generator;
a spark intensity sensor situated to sense intensity of sparks across the spark gap, the sensor being coupled to the controller;
wherein during operation the electrical energy supplied to the electrodes is sufficient to cause a plurality of sparks across the spark gap at intervals controlled by the controller, the controller is further configured to control energy supplied to the spark responsive to information received at least from the spark intensity sensor;
and wherein the spark energy is directed to enrich air with nitric oxide, the nitric oxide being produced from the air by the spark.

2. An inhaler as claimed in claim 1, wherein the spark intensity sensor comprises a pressure sensor, a pressure change sensor, a microphone, or any combination thereof.

3. An inhaler as claimed in claim 1, wherein spark intensity sensor is an electromagnetic sensor

4. An inhaler as claimed in claim 1, further comprising an input for receiving input from at least one blood parameter sensor, and wherein the controller adjusts the production of nitric oxide in response to the input from the blood parameter sensor.

5. An inhaler as claimed in claim 4, wherein the blood parameter sensor is an oximeter.

6. An inhaler as claimed in claim 4, wherein the blood parameter sensor comprises a methemoglobin sensor.

7. An inhaler as claimed in claim 1, wherein the controller utilizes information from the spark intensity sensor to estimate the condition of the at least one electrode.

8. An inhaler as claimed in claim 1, further comprising at least one treatment profile, wherein the controller is configured to control the inhaler according to the at least one treatment profile.

9. An inhaler as claimed in claim 8, wherein the profile comprises at least one element of a list of elements consisting of blood oxygen level, nitric oxide quantity per treatment, nitric oxide delivery rate per unit time, nitric oxide generation profile per breath cycle, nitric oxide generation responsive to information about one or more patient parameters, treatment duration, treatment cycle, nitric oxide generation responsive to environmental parameters, nitric oxide generation responsive to airflow in spark chamber, and any combination thereof.

10. An inhaler as claimed in claim 1, further comprising an inhalation sensor disposed to sense inhalation by a patient receiving the nitric oxide produced by the sparks, wherein the production of sparks occurs responsive to input from the inhalation sensor.

11. An inhaler as claimed in claim 1, further comprising a data link.

12. A inhaler as claimed in claim 11, wherein the data link is utilized for at least one of: programming the inhaler, controlling the inhaler, providing information to the inhaler, obtaining information from the inhaler, and any combination thereof.

13. An inhaler as claimed in claim 11, wherein at least a portion of the controller is remote to the inhaler.

14. An inhaler as claimed in claim 1, further comprising an inhalation sensor for sensing a breathing cycle of a patient using the inhaler, the breathing cycle comprising an inhalation and an exhalation, wherein the controller is configured to produce greater amounts of NO at the initial stage of the inhalation than NO production as the inhalation progresses.

15. An inhaler as claimed in claim 1, further comprising an air velocity sensor disposed to sense air velocity within the spark chamber, the air velocity sensor being coupled to the controller, wherein the controller is being configured to adjust the spark energy responsive to the velocity of air within the spark chamber.

16. An air and Nitric Oxide mixture inhaler having an input and an output, the input being in communication with air, the inhaler comprising:
a spark chamber;
at least two electrodes disposed within the spark chamber, the space between the electrodes forming a first spark gap;
a spark generator electrically coupled to the spark gap, the spark generator being capable of supplying controlled amount of electrical energy to the spark gap;
a controller coupled to the spark generator;
an input for receiving input from an at least one blood parameter sensor, and wherein the controller adjusts the production of nitric oxide in response to the input from the blood parameter sensor;
wherein during operation the electrical energy supplied to the electrodes is sufficient to cause a plurality of sparks across the spark gap at intervals controlled by the controller, the controller is further configured to control energy supplied to the spark gap responsive to information received at least from the blood parameter sensor;
and wherein the spark energy is directed to enrich air with nitric oxide, the nitric oxide being produced from the air by the spark.

17. An inhaler as claimed in claim 16 wherein the blood parameter sensor is an oximeter.

18. An inhaler as claimed in claim 16 wherein the blood parameter sensor is a methemoglobin sensor.

19. An inhaler as claimed in claim 16, further comprising a data link.

20. An inhaler as claimed in claim 19, wherein the blood parameter sensor is coupled to the inhaler via the data link.

21. An inhaler as claimed in claim 16, wherein at least a portion of the controller is remote to the inhaler.

22. An inhaler as claimed in claim 16, further comprising an inhalation sensor for sensing a breathing cycle of a patient using the inhaler, the breathing cycle comprising an inhalation and an exhalation, wherein the controller is configured to produce greater amounts of NO at the initial stage of the inhalation than NO production as the inhalation progresses.

23. An inhaler as claimed in claim 16, further comprising an air velocity sensor disposed to sense air velocity within the spark chamber, the air velocity sensor being coupled to the controller, wherein the controller is being configured to adjust the spark energy responsive to the velocity of air within the spark chamber.

24. An air and Nitric Oxide mixture inhaler having an input and an output, the input being in communication with air, the inhaler comprising:
a spark chamber;
at least two electrodes disposed within the spark chamber, the space between the electrodes forming a first spark gap;
a spark generator electrically coupled to the spark gap, the spark generator being capable of supplying controlled amount of electrical energy to the spark gap;
a controller coupled to the spark generator;
a spark intensity sensor situated to sense intensity of sparks across the spark gap, the sensor being coupled to the controller;
an input for receiving input from an at least one blood parameter sensor, and wherein the controller adjusts the production of nitric oxide in response to the input from the blood parameter sensor;
wherein during operation the electrical energy supplied to the electrodes is sufficient to cause a plurality of sparks across the spark gap at intervals controlled by the controller, the controller is further configured to control energy supplied to the spark gap responsive to information received at least from the blood parameter sensor, and from the spark intensity sensor;
and wherein the spark energy is directed to enrich air with nitric oxide, the nitric oxide being produced from the air by the spark.

25. An inhaler as claimed in claim 22, further comprising a data link.
